(19) United States
(12) Patent Application Publication
(10) Pub. No.: US 2026/0027031 A1
(43) Pub. Date: Jan. 29, 2026

(54) COSMETIC COMPOSITION COMPRISING NIACINAMIDE

(71) Applicant: LG H&H CO., LTD., Seoul (KR)

(72) Inventors: HWAN-IL CHO, Seoul (KR); Seung Heon YEON, Seoul (KR); Yoo Kyung HUR, Seoul (KR); Soon Sik KWON, Seoul (KR); Jong Sub HAN, Seoul (KR)

(73) Assignee: LG H&H CO., LTD., Seoul (KR)

(21) Appl. No.: 19/281,269

(22) Filed: Jul. 25, 2025

(30) Foreign Application Priority Data
Jul. 26, 2024 (KR) 10-2024-0099524
May 27, 2025 (KR) 10-2025-0069007

(51) Int. Cl. A61K 8/67; A61K 8/42; A61K 8/44; A61K 8/60; A61Q 19/00; A61Q 19/02; A61Q 19/08

(52) U.S. Cl. CPC A61K 8/675; A61K 8/42; A61K 8/44; A61K 8/602; A61Q 19/007; A61Q 19/02; A61Q 19/08; A61K 2800/72

(57) ABSTRACT

The present invention relates to a cosmetic composition comprising niacinamide.

By containing a high concentration of niacinamide, the present invention can provide a hypoallergenic cosmetic composition that exhibits outstanding effects in skin brightening, skin moisturizing, anti-aging, and/or pore refining, while significantly reducing skin irritation.

### CROSS-REFERENCE TO RELATED APPLICATION

This application claims priority to and the benefit of Korean Patent Application No. 2024-0099524, filed on Jul. 26, 2024, and Korean Patent Application No. 2025-0069007, filed on May 27, 2025, the disclosures of which are incorporated herein by reference in their entirety.

### BACKGROUND

### 1. Field of the Invention

The present invention relates to a cosmetic composition comprising niacinamide.

### 2. Discussion of Related Art

Niacinamide is a type of niacin belonging to the vitamin B group, also known as nicotinamide or pyridine-3-carboxylic acid, and is a water-soluble form of vitamin B3. Niacinamide, along with ceramide, simultaneously promotes the synthesis of cholesterol and fatty acids necessary for the formation of lamellar structures, thereby reducing moisture loss in the epidermis and increasing intercellular lipid synthesis, which is beneficial for skin moisturization. This niacinamide is essential for the human body and is also found in foods such as green vegetables and cereals. The importance of niacinamide for healthy skin has been recognized since the early 20th century. In particular, the skin brightening effect of niacinamide (Br J Dermatol. 2002 July; 147(1): 20-31), the moisturizing effect due to skin barrier improvement (J Cosmet Dermatol. 2004 April; 3(2): 88-93), the anti-aging effect that reduces wrinkles and improves elasticity (Int J Cosmet Sci. 2004 October; 26(5): 231-8), and the pore refining effect (Journal of Cosmetic and Laser Therapy. 2006; 8: 96-1010) have been reported.

As the cosmetic industry advances, the development of high-performance cosmetics is continuously taking place. Recently, as more consumers demand specific effects such as skin brightening, wrinkle reduction, elasticity, and pore refinement, the value of functional cosmetics with enhanced active ingredients has increased further. In particular, water-soluble vitamin B3, that is, niacinamide, as mentioned above, is widely used in cosmetics due to its various effects on the skin, and recently, many cosmetic products containing at least 10% niacinamide have been launched to further enhance its efficacy. However, when niacinamide is simply blended at a high concentration into a cosmetic formulation, niacinamide can cause neuro-sensory irritation such as itching, stinging, and burning sensations. As a result, there are difficulties in developing cosmetics containing high concentrations of niacinamide.

To reduce the irritation caused by niacinamide, various additional ingredients have been introduced. For example, skin irritation relievers containing green tea root extract as an active ingredient (Korean Registered Patent No. 10-2224350) and hypoallergenic skin brightening cosmetic compositions containing arbutin, niacinamide, gentiana extract, alpha-bisabolol, beta-glucan, panthenol, green tea extract, centella asiatica extract, arnica flower extract, Achillea millefolium extract, and trehalose as active ingredients (Korean Registered Patent No. 10-2015173) have been developed.

However, the skin irritation caused by niacinamide does not appear in a single form, but rather as various neuro-sensory responses, such as itching, stinging, and burning. Therefore, the use of only one anti-inflammatory ingredient is often insufficient to alleviate this irritation. Accordingly, to deliver the potent effects of niacinamide, it is necessary to develop cosmetics that can suppress neuro-sensory irritation and reduce niacinamide-induced skin irritation, even when niacinamide is contained at a high concentration of 10% or more.

To address the issue of skin irritation, the inventors discovered that skin irritation can be alleviated by using one or more compounds selected from sarcosine, panthenol, and hydroxyphenylpropamidobenzoic acid, together with troxerutin, as irritation relievers, and based on this discovery, the present invention was completed.

### SUMMARY OF THE INVENTION

The present invention is directed to providing a hypoallergenic cosmetic composition that contains a high concentration of niacinamide while significantly reducing skin irritation and neuro-sensory irritation.

According to an aspect of the present invention, there is provided a cosmetic composition comprising:
(A) niacinamide;
(B) troxerutin; and
(C) one or more compounds selected from the group consisting of sarcosine, panthenol, and hydroxyphenylpropamidobenzoic acid.

According to one aspect of the present invention, there is provided a method for reducing skin irritation or neuro-sensory irritation caused by cosmetic composition comprising a high concentration of niacinamide, particularly in an amount of 10 wt % or more, by adding troxerutin along with one or more of sarcosine, panthenol, and hydroxyphenylpropamidobenzoic acid to the composition.

### DETAILED DESCRIPTION OF EXEMPLARY EMBODIMENTS

The present invention relates to a cosmetic composition comprising:
(A) niacinamide;
(B) troxerutin; and
(C) one or more compounds selected from the group consisting of sarcosine, panthenol, and hydroxyphenylpropamidobenzoic acid.

Hereinafter, the composition of the present invention will be described in detail.

In addition, when a part is described as “comprising” a certain component, this does not mean that other components are excluded, but rather that other components may also be added unless specifically stated to the contrary.

In the prevent invention, (A) niacinamide is a type of niacin belonging to the vitamin B group, and when included in the cosmetic composition, it may exhibit effects such as skin brightening, moisturizing, anti-aging, and pore refining.

In a specific example, the content of (A) niacinamide may be 10 wt % or more based on the total weight (100 wt %) of the cosmetic composition, and the upper limit may be 50 wt % or less. Specifically, the content may be 10 to 50 wt %, 10 to 30 wt %, 10 to 25 wt %, 10 to 20 wt %, 12 to 50 wt %, 12 to 30 wt %, 12 to 25 wt %, 12 to 20 wt %, 15 to 50 wt %, 15 to 30 wt %, 15 to 25 wt %, 15 to 20 wt %, or 10 to 20 wt %. When the content of niacinamide is 10 wt % or more, the skin improvement effects of niacinamide are excellent. When the content of niacinamide is 50 wt % or less, discomfort caused by the high concentration of niacinamide may be avoided.

In the present invention, (B) troxerutin is a semi-synthetic compound derived from rutinoside, a flavonoid obtained from the Japanese pagoda tree (Sophora japonica), and may inhibit factors that act on leukocytes and vascular endothelial cells, which are involved in the secretion of inflammatory mediators and inflammatory metabolic processes. Specifically, when applied topically, troxerutin acts as an antioxidant, providing effects such as the removal of reactive oxygen species and the prevention of oxidative stress. In the present invention, by including troxerutin and niacinamide as active ingredients, a synergistic effect may be achieved in alleviating skin irritation and providing anti-inflammatory effects.

In a specific example, the content of (B) troxerutin may be 0.1 to 1.5 wt %, 0.1 to 1.5 wt %, 0.3 to 1.3 wt %, or 0.5 to 1.2 wt %, based on the total weight (100 wt %) of the cosmetic composition. Within these ranges, the effect of alleviating skin irritation may be achieved.

In the present invention, in addition to the aforementioned (A) niacinamide and (B) troxerutin, (C) compounds may be further included. The (C) compounds have anti-inflammatory effects and may provide enhanced hypoallergenic effects for the skin as irritation relievers.

In the present invention, the (C) compounds may include one or more selected from the group consisting of sarcosine, panthenol, and hydroxyphenylpropamidobenzoic acid. Specifically, two or more may be included, and all three components may be included together.

In a specific example, the content of the (C) compound may be 0.01 to 5.0 wt %, 0.01 to 4.0 wt %, 0.01 to 3.5 wt %, 0.05 to 4.0 wt %, 0.05 to 3.5 wt %, or 0.1 to 3.0 wt %, based on the total weight (100 wt %) of the cosmetic composition.

In a specific example, based on the total weight (100 wt %) of the cosmetic composition, the content of sarcosine may be 0.05 to 0.5 wt %, 0.05 to 0.3 wt %, 0.1 to 0.3 wt %, or 0.15 to 0.25 wt %, the content of panthenol may be 1.0 to 5.0 wt %, 1.0 to 4.0 wt %, 1.0 to 3 wt %, 1.5 to 3.5 wt %, or 2.0 to 3.0 wt %, and the content of hydroxyphenylpropamidobenzoic acid may be 0.02 to 0.5 wt %, 0.02 to 0.3 wt %, 0.05 to 0.3 wt %, or 0.05 to 0.15 wt %.

The cosmetic composition of the present invention may further include a polyol as a moisturizer. The polyol may affect the precipitation stability and usability of niacinamide.

In a specific example, the polyol may include one or more selected from the group consisting of glycerin, dipropylene glycol, butylene glycol, pentylene glycol, methylpropanediol, sorbitol, diglycerin, erythritol, pentaerythritol, polybutylene glycol-10, polyglycerin-3, polyglycerin-4, polyglycerin-6, polyglycerin-10, polyglycerin-20, polyglycerin-40, sorbeth-5, sorbeth-6, sorbeth-20, sorbeth-30, sorbeth-40, inositol, maltitol, maltose, mannan, mannitol, mannose, lactitol, lactose, dihydroxypropyl PG-glucoside, dichiaoctanediol, fructose, glucamine, methylglucamine, and glucose.

In a specific example, the content of the polyol may be 10 to 40 wt % based on the total weight (100 wt %) of the cosmetic composition. When the content of the polyol is less than 10 wt %, there may be problems with precipitation stability, and when the content exceeds 40 wt %, precipitation stability may be improved but usability may be significantly reduced.

The cosmetic composition of the present invention may further include a skin improvement material.

In a specific example, the skin improvement material may be one or more selected from the group consisting of disodium phenyl dibenzimidazole tetrasulfonate, arbutin, magnesium ascorbyl phosphate, ascorbyl glucoside, butyl methoxydibenzoylmethane, bis-ethylhexyloxyphenol methoxyphenyl triazine, acetyl phytosphingosine, asiaticoside, ethyl ascorbyl ether, polyethoxylated retinamide, polydatin, kinetin, aminobutyric acid, oligopeptide-2, peony extract, bilberry extract, ginseng extract, and fermented sugarcane extract.

The cosmetic composition of the present invention may be prepared in any formulation commonly manufactured in the related art. For example, it may be formulated as a solution, suspension, emulsion, paste, gel, cream, lotion, emulsion foundation, spray, etc. More specifically, it may be prepared in the form of a toner, lotion, nourishing cream, massage cream, serum, essence, eye cream, pack, or spray.

The cosmetic composition according to the present invention may be used for alleviating skin irritation, skin brightening, skin moisturizing, anti-aging, and/or pore refining.

In another aspect, the present invention relates to a method for improving the skin condition of a subject in need thereof, comprising applying a cosmetic composition comprising:
(A) niacinamide;
(B) troxerutin; and
(C) one or more compounds selected from the group consisting of sarcosine, panthenol, and hydroxyphenylpropamidobenzoic acid.

In the present invention, the skin condition may include one or more of skin brightening, skin moisturizing, anti-aging, and pore refining.

In another aspect, the present invention relates to a method of manufacturing a hypoallergenic composition comprising 10% or more of niacinamide, comprising adding troxerutin and one or more of sarcosine, panthenol, and hydroxyphenylpropamidobenzoic acid into the composition comprising niacinamide.

Hereinafter, the present invention will be described in detail through examples and test examples to aid understanding of the present invention. However, these examples and test examples of the present invention may be modified in various ways, and the scope of the present invention should not be construed as being limited to these examples and test examples. The examples and test examples of the present invention are provided for illustrative purposes to more fully explain the present invention to those of ordinary skill in the art.

### EXAMPLES

### Examples 1 to 6 and Comparative Examples 1 to 4

Cosmetic compositions were prepared according to the ingredients and their contents (wt %) listed in Tables 1 and 2.

First, ingredients 1 to 7 were weighed into a beaker and uniformly dissolved using a disper mixer at a temperature of 45 to 50° C. Next, ingredients 8 to 13 were added, solubilized for 5 minutes, cooled, and deaerated to prepare a serum-type cosmetic containing a high concentration of niacinamide.

TABLE 1

| | Ingredients (wt %) | Example 1 | Example 2 | Example 3 | Example 4 | Example 5 | Example 6 |
|---|---|---|---|---|---|---|---|
| 1 | Purified water | To 100 | To 100 | To 100 | To 100 | To 100 | To 100 |
| 2 | Niacinamide | 15.0 | 15.0 | 15.0 | 15.0 | 15.0 | 15.0 |
| 3 | Butylene glycol | 10.0 | 10.0 | 10.0 | 10.0 | 10.0 | 10.0 |
| 4 | Propanediol | 5.0 | 5.0 | 5.0 | 5.0 | 5.0 | 5.0 |
| 5 | 1,2-Hexanediol | 1.5 | 1.5 | 1.5 | 1.5 | 1.5 | 1.5 |
| 6 | Polyacrylate | 0.15 | 0.15 | 0.15 | 0.15 | 0.15 | 0.15 |
| | crosspolymer-6 | | | | | | |
| 7 | Xanthan gum | 0.05 | 0.05 | 0.05 | 0.05 | 0.05 | 0.05 |
| 8 | Troxerutin | 1.0 | 1.0 | 1.0 | 1.0 | 1.0 | 1.0 |
| 9 | Panthenol | 2.5 | 0 | 0 | 2.5 | 0 | 2.5 |
| 10 | Sarcosine | 0 | 0.2 | 0 | 0.2 | 0.2 | 0.2 |
| 11 | Hydroxyphenylpropamidobenzoic | 0 | 0 | 0.1 | 0 | 0.1 | 0.1 |
| | acid | | | | | | |
| 12 | Allantoin | 0 | 0 | 0 | 0 | 0 | 0 |
| 13 | Portulaca oleracea extract | 0 | 0 | 0 | 0 | 0 | 0 |

TABLE 2

| | | Comparative | Comparative | Comparative | Comparative |
|---|---|---|---|---|---|
| | Ingredients (wt %) | Example 1 | Example 2 | Example 3 | Example 4 |
| 1 | Purified water | To 100 | To 100 | To 100 | To 100 |
| 2 | Niacinamide | 15.0 | 15.0 | 15.0 | 15.0 |
| 3 | Butylene glycol | 10.0 | 10.0 | 10.0 | 10.0 |
| 4 | Propanediol | 5.0 | 5.0 | 5.0 | 5.0 |
| 5 | 1,2-Hexanediol | 1.5 | 1.5 | 1.5 | 1.5 |
| 6 | Polyacrylate | 0.15 | 0.15 | 0.15 | 0.15 |
| | crosspolymer-6 | | | | |
| 7 | Xanthan gum | 0.05 | 0.05 | 0.05 | 0.05 |
| 8 | Troxerutin | 0 | 0 | 0 | 0 |
| 9 | Panthenol | 0 | 2.5 | 2.5 | 2.5 |
| 10 | Sarcosine | 0 | 0.2 | 0.2 | 0.2 |
| 11 | Hydroxyphenyl- | 0 | 0 | 0 | 0 |
| | propamidobenzoic | | | | |
| | acid | | | | |
| 12 | Allantoin | 0 | 0 | 0.2 | 0 |
| 13 | Portulaca oleracea | 0 | 0 | 0 | 2.0 |
| | extract | | | | |

### Experimental Example 1. Skin Irritation Evaluation 1: Human Closed Patch Test

The skin irritation of the cosmetic compositions prepared in Examples 1 to 6 and Comparative Examples 1 to 4 was evaluated. The evaluation was performed using the human closed patch test method, which enables the comparative evaluation of multiple samples at the same time.

The evaluation was performed on 30 healthy adult men and women in accordance with the CTFA guidelines (The Cosmetic, Toiletry and Fragrance Association. Inc., Washington, D.C., 20036, 1991). Specifically, 25 μL of each sample was applied to a Finn chamber, which was then affixed to the test area on the subject's back with tape. After 48 hours of application, the patch was removed, and after an additional 2 hours, the skin reaction at the test site was evaluated according to the criteria below. The skin irritation index was then calculated according to Equation 1, and the results are shown in Table 3.

### <Evaluation Criteria>

−: No erythema or unusual reaction
±: Slight redness compared to the surrounding area
+: Marked redness compared to the surrounding area
++: Severe redness and swelling compared to the surrounding area

$$Skin irritation index = [ \{ ( Number of \pm ) \times 1 \} + \{ ( Number of + ) \times 2 \} + \{ ( Number of ++ ) \times 3 \} ] / Number of test subjects \qquad < Equation 1 >$$

TABLE 3

| | Number of | Evaluation results | | | | Skin irritation |
|---|---|---|---|---|---|---|
| Composition | test subjects | ++ | + | ± | − | index |
| Example 1 | 30 | 0 | 1 | 3 | 26 | 0.17 |
| Example 2 | 30 | 0 | 2 | 3 | 25 | 0.23 |
| Example 3 | 30 | 0 | 1 | 4 | 25 | 0.20 |
| Example 4 | 30 | 0 | 0 | 3 | 27 | 0.10 |
| Example 5 | 30 | 0 | 0 | 2 | 28 | 0.07 |
| Example 6 | 30 | 0 | 0 | 0 | 30 | 0.00 |
| Comparative | 30 | 1 | 5 | 5 | 19 | 0.60 |
| Example 1 | | | | | | |
| Comparative | 30 | 1 | 4 | 4 | 21 | 0.50 |
| Example 2 | | | | | | |
| Comparative | 30 | 0 | 3 | 5 | 22 | 0.37 |
| Example 3 | | | | | | |
| Comparative | 30 | 0 | 3 | 4 | 23 | 0.33 |
| Example 4 | | | | | | |

As shown in Table 3, when a high concentration of niacinamide was used without (C) compounds as in Comparative Example 1, it can be seen that skin irritation significantly increased. In contrast, when one or more compounds selected from sarcosine, panthenol, and hydroxyphenylpropamidobenzoic acid were combined with troxerutin, as in Examples 1 to 6, skin irritation was significantly reduced. In addition, when two or more of these compounds were used in combination, no skin irritation was observed.

Additionally, as seen in Comparative Examples 2 to 4, when sarcosine and panthenol were applied without troxerutin, skin irritation was also found to be significantly increased. These results demonstrate that combining one or more of sarcosine, panthenol, and hydroxyphenylpropamidobenzoic acid with troxerutin provides a hypoallergenic effect on the skin.

### Experimental Example 2. Skin Irritation Evaluation 2: Cumulative Patch Test

A cumulative patch test that can more precisely observe differences in skin irritation was conducted to evaluate the skin irritation of the cosmetic compositions of Examples 2 to 6 and Comparative Examples 1, 3, and 4.

The evaluation was performed by applying 25 mg of the cosmetic composition to the inner fold of the elbow of 20 subjects (adult men and women). Specifically, the cosmetic composition was applied twice daily (at 9 a.m. and 7 p.m.) for seven days, and the condition of the skin was checked each morning before application. Subsequently, neuro-sensory irritation over time was assessed through a subject questionnaire, according to the criteria in Table 4, and the results were used to calculate the skin irritation index based on Equation 2. The results thereof are shown in Table 5 below.

TABLE 4

| Irritation | Irritation | |
|---|---|---|
| degree | response | |
| (Score) | (Reaction) | Evaluation criteria |
| 0 | − | No symptoms |
| 1 | +− | Slight neuro-sensory irritation |
| 2 | + | Mild neuro-sensory irritation |
| 3 | ++ | Distinct neuro-sensory irritation |
| 4 | +++ | Severe neuro-sensory irritation |

$$Skin irritation index = Degree of irritation \times ( Number of subjects showing a response / Total number of subjects ) \qquad [ Equation 2 ]$$

TABLE 5

| | Neurosensory irritation test (Skin irritation index) | | | | |
|---|---|---|---|---|---|
| Composition | Day 1 | Day 2 | Day 3 | Day 4 | Day 7 |
| Example 2 | 0.10 | 0.05 | 0.05 | 0.10 | 0.10 |
| Example 3 | 0.10 | 0.10 | 0.10 | 010 | 0.15 |
| Example 4 | 0.05 | 0.00 | 0.05 | 0.05 | 0.10 |
| Example 5 | 0.00 | 0.00 | 0.05 | 0.05 | 0.00 |
| Example 6 | 0.00 | 0.00 | 0.00 | 0.05 | 0.00 |
| Comparative | 0.30 | 0.18 | 0.21 | 0.21 | 0.18 |
| Example 1 | | | | | |
| Comparative | 0.28 | 0.15 | 0.15 | 0.18 | 0.18 |
| Example 3 | | | | | |
| Comparative | 0.25 | 0.20 | 0.20 | 0.20 | 0.20 |
| Example 4 | | | | | |

As shown in Table 5, when one or more of sarcosine, panthenol, and hydroxyphenylpropamidobenzoic acid were combined with troxerutin (Examples 2 to 6), no concerning skin irritation index was observed even at a niacinamide concentration of 15%. In contrast, when (C) compounds were not used (Comparative Example 1), or when sarcosine and panthenol were applied without troxerutin (Comparative Examples 3 and 4), significant neuro-sensory irritation was observed.

### Experimental Example 3. Evaluation of Pore-Refining Effect

The pore-refining effect of the hypoallergenic cosmetic composition containing a high concentration of niacinamide, prepared in Example 6, was evaluated in vivo.

The evaluation was conducted on a total of 31 adult men and women aged 20 to 67. First, immediately before the start of the evaluation, the area on the left cheek beside the nose of each subject was measured using Antera 3D. The volume (representing pore volume), affected area (representing pore area), and count (number of pores) were recorded. Next, the subjects were instructed to apply the cosmetic composition to the facial area twice daily, in the morning and evening, after washing their faces. In this manner, the cosmetic was used for two weeks. Afterward, the same area on the left cheek beside the nose was measured again using Antera 3D, and the changes before and after use of the cosmetic composition were analyzed. A decrease in the measured values indicated an improvement in pore condition, and the calculated improvement rates (%) were presented in Table 6.

TABLE 6

| Pore volume | Pore area | Pore count |
|---|---|---|
| improvement | improvement | improvement |
| rate (%) | rate (%) | rate (%) |
| 28.55 | 24.77 | 18.32 |

As shown in Table 6, it can be seen that the use of the hypoallergenic cosmetic composition containing a high concentration of niacinamide, prepared according to the present invention, resulted in a significant pore-refining effect.

The cosmetic composition according to the present invention contains a high concentration of niacinamide, thereby exhibiting excellent effects in skin brightening, skin moisturizing, and/or anti-aging, as well as remarkable improvements in pore conditions and skin tone.

In addition, the cosmetic composition according to the present invention is suitable as a hypoallergenic cosmetic composition, as it significantly reduces the skin irritation caused by the high concentration of niacinamide.

What is claimed is:

1. A cosmetic composition comprising:
(A) niacinamide;
(B) troxerutin; and
(C) one or more compounds selected from the group consisting of sarcosine, panthenol, and hydroxyphenylpropamidobenzoic acid.

2. The cosmetic composition of claim 1, wherein the content of (A) niacinamide is 10 to 20 wt % based on the total weight of the cosmetic composition.

3. The cosmetic composition of claim 1, wherein the content of (B) troxerutin is 0.1 to 1.5 wt % based on the total weight of the cosmetic composition.

4. The cosmetic composition of claim 1, wherein the content of the (C) compound is 0.01 to 5.0 wt % based on the total weight of the cosmetic composition.

5. A method for improving the skin condition of a subject in need thereof, comprising applying a cosmetic composition comprising:
(A) niacinamide;
(B) troxerutin; and
(C) one or more compounds selected from the group consisting of sarcosine, panthenol, and hydroxyphenylpropamidobenzoic acid.

6. The method of claim 5, wherein the skin condition is one or more of skin brightening, skin moisturizing, anti-aging, and pore refining.

7. A method of manufacturing a hypoallergenic composition comprising 10% or more of niacinamide,
comprising adding troxerutin and one or more of sarcosine, panthenol, and hydroxyphenylpropamidobenzoic acid into the composition comprising niacinamide.
